(19) United States
(12) Patent Application Publication
(10) Pub. No.: US 2020/0345659 A1
(43) Pub. Date: Nov. 5, 2020

(54) TOPICAL ANALGESIC SPRAY COMPOSITIONS

(71) Applicant: Bayer HealthCare LLC, Indianola, PA (US)

(72) Inventors: Debanjan DAS, Morristown, NJ (US); Reginald BRADLEY, Bedminster, NJ (US); Eric DANN, Safety Harbor, FL (US); Thomas DANN, Palm Harbor, FL (US); Courtney C. HAYNES, Dunedin, FL (US); Gerard MEISEL, Budd Lake, NJ (US); Renee NELSON, Brandon, FL (US); Soundarya VAITHIANATHAN, Parsippany, NJ (US); Emanuel VIZZOTTI, Millburn, NJ (US); Reinhard WALTER, Morristown, NJ (US)

(21) Appl. No.: 16/856,171

(22) Filed: Apr. 23, 2020

Related U.S. Application Data
(60) Provisional application No. 62/983,263, filed on Feb. 28, 2020.
(60) Provisional application No. 62/841,105, filed on Apr. 30, 2019.
(60) Provisional application No. 62/983,263, filed on Feb. 28, 2020.

(51) Int. Cl. A61K 31/125; A61K 31/045; A61K 9/00; A61K 47/34; A61K 47/44; A61K 47/10; A61K 9/12

(52) U.S. Cl. CPC A61K 31/125; A61K 31/045; A61K 9/0014; A61K 9/12; A61K 47/44; A61K 47/10; A61K 47/34

(57) ABSTRACT

Provided are topical analgesic spray compositions and topical analgesic spray concentrates containing menthol and camphor in high concentrations. The present disclosure also provides organoleptic compositions for use in the topical analgesic spray compositions and concentrates to provide enhanced sensory experience and long-lasting pain-relief.

### FIELD OF THE INVENTION

The present disclosure relates to topical analgesic compositions, and more particularly to topical analgesic spray compositions comprising menthol and camphor.

### BACKGROUND OF THE INVENTION

Menthol and camphor are often provided together in topical analgesic formulations to help treat musculoskeletal injuries and disorders, including pulled muscles, sprained muscles, and arthritis. Although many mentholated and camphorated medications are available for consumer use, there is interest in developing spray formulations having improved sensory properties and/or containing even higher concentrations of menthol and camphor than are currently present in existing products. Highly-concentrated mentholated and camphorated formulations would allow consumers to achieve overall longer-lasting pain relief while requiring fewer applications of the formulation.

However, the physical properties of menthol and camphor present a unique set of challenges for preparing topical analgesic compositions, especially at high concentrations. Both are oleaginous and solid at room temperature, which encumbers the development of highly-concentrated mentholated- and camphorated-formulations having smooth, non-greasy skin feel and other properties (such as scent) that are acceptable to consumers. Topical formulations must solubilize menthol and camphor while also providing the necessary characteristics to enable application to the skin and afford an enjoyable sensory experience to the consumer.

This balance is especially challenging to achieve in topical spray medications, for which the formulations must also be aerosolized. Even at low concentrations of menthol and camphor, there are many difficulties associated with spraying oily components in an aerosol form. Achieving consistent flow and uniform spray distribution with minimal loss of active ingredients to volatilization are some of the properties required for aerosol sprays. Moreover, it is desirable to have topical spray medications that are quick-drying and are require minimal effort to apply, i.e., no-rub. The accommodation of higher concentrations of menthol and camphor adds to the complexity of achieving these sensory and engineering elements in a single topical spray formulation.

Consequently, the successful preparation of topical analgesic sprays containing high concentrations of menthol and camphor and also having favorable tactile properties is not trivial. There remains a need for alternative topical analgesic spray formulations comprising menthol and camphor, and, in particular, analgesic spray compositions having high concentrations of menthol and camphor.

### SUMMARY OF THE INVENTION

Provided herein are topical analgesic spray compositions comprising menthol and camphor, and methods of preparing topical analgesic spray compositions comprising menthol and camphor. More specifically, provided herein are topical analgesic spray compositions that include and more than 2 wt. % menthol and more than 1 wt. % camphor. Also provided herein are topical analgesic spray concentrates that include more than 10 wt. % menthol and more than 5 wt. % camphor and that may be used to prepare the topical analgesic spray compositions of the present disclosure.

Conventional topical analgesic spray compositions containing menthol and/or camphor most commonly employ water and an alcohol as co-solvents to solubilize the active ingredients. Typically, these formulations are not directly mixed with any propellants. Instead, these conventional spray formulations are administered by a manual spray pump mechanism or a bag-on-valve aerosol system. In the manual spray pump mechanism, the consumer applies manual force to a positive displacement pump that draws the spray compositions into a siphon tube and forces the liquid formulation through a nozzle to form a spray. In the bag-on-valve technology, the spray formulation is placed in the interior of a bag inside a pressurized can. A propellant is also provided inside the pressurized can on the exterior of the bag and is separated from the formulation by the bag. When the dispenser valve is depressed in the bag-on-valve system, the propellant provides positive pressure to displace the spray formulation inside the bag and force the formulation through a nozzle to form a spray.

However, topical analgesic spray formulations containing water and using these pump mechanisms or bag-on-valve systems do not dry quickly. Typically they remain wet, drip from the area of application, may require the consumer to manually rub the formulation into the skin, and often leave the consumer with a greasy or tacky feeling on their skin and hands. Wet sprays also have the potential to wet or stain clothing that a person is wearing. In addition, traditional formulations using water and an alcohol as co-solvents have limited solubility for menthol and camphor and are unable to support high concentrations of menthol and camphor in spray formulations.

As described herein, high concentrations of menthol and camphor in a spray composition can be achieved by combining the menthol and camphor in spray concentrate containing a relatively large quantity of alcohol as primary solvent (e.g., from 50 to 70 wt. %), and further combining the spray concentrate directly with a high proportion of propellants (e.g., more than 75 wt. % of one or more propellants). By utilizing an alcohol solvent and a high proportion of propellants directly mixed with menthol and camphor, a relatively high payload of menthol and camphor has been achieved in the topical analgesic spray compositions disclosed. Moreover, as a result of the large proportion of the solvent and propellants present and their rapid evaporation, the topical analgesic spray compositions are quick-drying (within 20 seconds after application) and feel smooth on the skin.

Additionally, once applied to a user's skin, topical analgesic spray compositions provided herein develop an encapsulating matrix in the form of a film layer on the skin. In particular, once the primary solvent of the product evaporates, a film layer is formed on the area of application. The film layer comprises an encapsulating matrix that traps fragments or deposits of the active ingredients. This encapsulating matrix is caused by a phase change of the specific polymer system used once the formulation is applied on the skin, which is described in more detail below. The combination of the encapsulating matrix and the polymer system achieves sustained delivery of actives on the skin by being wash-resistant.

Accordingly, by using a large quantity of an alcohol solvent, directly mixing the active ingredients with a large quantity of propellants, and incorporating a film-forming polymer agent, sustained delivery of high concentrations of menthol and camphor has been achieved in the topical analgesic spray compositions disclosed.

In one aspect, provided herein is a topical analgesic spray composition, comprising: 2 to 4 wt. % menthol; 1 to 3 wt. % camphor, 10 wt. % or more ethanol; and 75 wt. % or more of one or more propellants.

In another aspect, provided herein is an aerosol spray dispenser, comprising a topical analgesic spray composition, wherein the topical analgesic spray composition comprises: 2 to 4 wt. % menthol; 1 to 3 wt. % camphor, 10 wt. % or more ethanol; and 75 wt. % or more of one or more propellants.

In one aspect, the present disclosure provides a method for treating muscle and joint ache or pain, comprising administering to a patient in need thereof an analgesic spray composition.

In yet another aspect, provided herein is a topical analgesic spray concentrate comprising: 10 to 16 wt. % menthol; 5 to 11 wt. % camphor; 0.05 to 1 wt. % film-forming agent; and 50 to 70 wt. % ethanol.

In still yet another aspect, the present disclosure provides a method for treating muscle and joint ache or pain, comprising administering to a patient in need thereof an analgesic spray concentrate.

In one aspect, provided herein is A method of preparing a topical analgesic spray composition, the method comprising: preparing a mixture comprising a solvent and a film-forming agent; adding menthol and camphor to the mixture comprising the solvent and the film-forming agent to form a topical analgesic spray concentrate; and combining the topical analgesic spray concentrate with one or more propellants to provide the topical analgesic spray composition, wherein the topical analgesic spray composition comprises 75 wt. % or more of one or more propellants.

### DETAILED DESCRIPTION OF THE INVENTION

Described herein are topical analgesic spray compositions comprising menthol and camphor, methods for preparing the topical spray analgesic compositions and methods of using the topical spray analgesic compositions.

Provided herein is a topical analgesic spray composition comprising menthol and camphor, and, more particularly, a topical analgesic spray composition having high concentrations of menthol and camphor. When applied to the skin, the topical analgesic spray compositions of the present disclosure, among other favorable sensory characteristics, possess minimal drying time (quick-drying and no-drip), do not require any manual effort in application (i.e., no-rub), dry clear on the skin, and provide a long-lasting pain-relieving effect. Highly-concentrated menthol and camphor spray compositions having these attributes are achieved in the present disclosure by combining menthol and camphor with a volatile alcoholic solvent and relatively large quantity of hydrocarbon propellants.

It has been discovered that high concentrations of menthol and camphor can be delivered as an aerosolized spray when admixed with a suitably solubilizing, quickly evaporating solvent, such as ethanol, and a high proportion of hydrocarbon propellants, particularly isopentane, while still retaining the desired skin feel and other sensory properties. The solvent and hydrocarbon propellants work together to confer the necessary aerosol properties to the highly-concentrated menthol/camphor spray formulations described herein as well as some beneficial sensory properties (quick-drying, no-drip). The alcoholic solvent balances a high solubility for both menthol and camphor in the formulation with a relatively low surface tension and relatively high vapor pressure as compared to water. These properties of the solvent enable the spray formulation to disperse as fine, evenly distributed, and uniformly concentrated droplets of menthol and camphor when dispensed from an aerosol container. Moreover, once the aerosol droplets reach the surface of the skin, the solvent is readily evaporated, leaving the active ingredients as a dry film. The propellants also facilitate the formation of the aerosol spray. In the topical analgesic formulations described herein, the hydrocarbon propellants are directly combined with the spray formulation. The propellant mixture itself provides the majority weight percentage of the overall topical analgesic spray composition. In particular, the spray compositions of the present disclosure combine the spray formulation with a large proportion of a propellant mixture containing a majority of isopentane as a primary propellant as well as secondary propellants isobutane and propane in a lower concentration. Upon being dispensed, the force of the flash evaporation of the propellants provides the even distribution of the aerosol, thereby preventing aggregation or formation of large droplets and, thus, also contributing to the quick-drying nature of the spray.

In another aspect, provided herein is a topical analgesic spray concentrate that, when admixed with hydrocarbon propellants as described herein, produces the analgesic spray composition having high concentration of menthol and camphor. As described herein, the topical analgesic spray concentrate refers to the base formulation of the topical analgesic spray composition, containing all components of the corresponding topical analgesic spray composition including the solvent, but excluding the hydrocarbon propellants. It should be understood that a topical analgesic spray concentrate will contain higher concentrations of the ingredients (by weight percentage) than the corresponding topical analgesic spray composition due to the absence of the propellant in the concentrate. It should be further recognized that the propellants of the topical analgesic spray compositions will evaporate during application to the consumer or patient's skin. As such, the concentrations of menthol and camphor, and other actives immediately arriving at the site of skin upon application are those provided in the concentrate.

The topical analgesic spray formulations described herein also contain certain film-forming agents that contribute to the long-lasting pain relieving effect of the spray compositions. It has been unexpectedly found that the use of certain film-forming agents in very low amounts in the topical analgesic spray compositions described herein augments the sensation of long-lasting relief by forming an encapsulating matrix on the skin. The encapsulating matrix localizes and traps deposits of the active ingredients when applied to the skin, prevents flashing off with the propellants upon actuation and adds substantive properties to hold the actives in the site of application, thereby resulting in a slow-release of the active ingredients to produce a long-lasting analgesic effect.

In some embodiments, the encapsulating matrix film develops due to the combination of a unique polymer system and a phase change phenomenon. The particular polymer system is explained in detail below. This polymer system is soluble in alcohol and can remain solubilized in the alcohol along with the active(s). Once a topical analgesic composition is applied to the skin, the solvent (i.e., alcohol) evaporates. Simultaneously, the topical analgesic composition is exposed to moisture present on the skin and moisture produced by the skin. Thus, the topical analgesic applied on the skin changes from an alcohol-based composition to a water-based composition. In some embodiments, because the polymer system is less soluble in water than in alcohol, it forms a film, encapsulating the active(s). This encapsulating matrix can hold, or localize, the active(s) at the area of application to provide sustained delivery or a prolonged benefit to the area of skin. The encapsulating matrix is also wash-resistant.

Additionally, topical analgesic spray compositions according to embodiments provided herein may include both menthol and camphor. The combination of menthol and camphor can produce an oily eutectic mixture which can have a limited solubility in most solvents commonly used in topical analgesics (and specifically topical analgesic compositions for roll-on applicators). However, in some embodiments, topical analgesic spray concentrates can include a high payload of up to 35 wt. % menthol-camphor solution.

In yet another aspect, the present disclosure provides an organoleptic composition that may be incorporated into the topical analgesic spray compositions and topical analgesic spray concentrates containing menthol and camphor. The organoleptic compositions described herein contain a selection of cooling and warming sensates and essential oil mixture. It has been further discovered that particular combinations of cooling and warming sensates, along with a selection of essential oils, complement the sensory effects provided by the menthol and camphor, and thus also contribute to the consumer's impression of long-lasting relief from pain and aches.

Provided below is a discussion of topical analgesic spray compositions, topical analgesic spray concentrates, topical analgesic spray compositions as provided in an aerosol spray dispenser, and methods for preparing topical analgesic spray compositions and concentrates.

### Topical Analgesic Spray Compositions and Concentrates

Topical analgesic spray concentrates and compositions according to embodiments provided herein include an active ingredient or ingredients (e.g., menthol, camphor), a solvent, a film-forming agent, an emollient, a thinning agent, and fragrance, and, in the topical analgesic spray compositions, one or more propellants. In some embodiments, topical analgesic spray concentrates and compositions comprise an organoleptic composition. An organoleptic composition may comprise cooling and warming sensates, an essential oil mixture comprising one or more essential oils, vitamin E, linseed oil, and optionally also further excipients. Organoleptic compositions are described in detail further below.

### Active Ingredients

As described herein, the topical analgesic spray concentrates and topical analgesic spray compositions of the present disclosure comprise menthol and camphor.

In some embodiments, the topical analgesic spray concentrate and topical analgesic spray composition may include menthol. Menthol can be naturally obtained from the oils of corn mint, peppermint, and other mints, or can be obtained as a synthetic product. Menthol is commonly used in topical analgesics because it has local anesthetic (i.e., a medication that causes the absence of pain sensations) and counterirritant (i.e., a substance that creates irritation or mild inflammation in one location to lessen discomfort or inflammation in a second location) properties. In some embodiments, the topical analgesic spray concentrate comprises from 1 wt. % to 16 wt. % menthol, from 5 wt. % to 16 wt. % menthol, or from 10 wt. % to 16 wt. % menthol. In certain embodiments, the topical spray concentrate comprises from 10 wt. % to 16 wt. % menthol. In certain embodiments, the spray concentrate comprises 16 wt. % menthol. In other embodiments the topical analgesic spray composition comprises from 0.2 wt. % to 4 wt. %, from 0.2 wt. % to 3.2 wt. % menthol, from 1 wt. % to 3.2 wt. % menthol, from 2 wt. % to 3.2 wt. %, or from 2 wt. % to 4 wt. % menthol. In certain embodiments, the topical analgesic spray composition comprises 3.2 wt. % menthol.

In some embodiments, the topical analgesic spray concentrate and topical analgesic spray composition may include camphor. Camphor is a terpenoid found in the wood of camphor laurel, an evergreen tree, and kapur tree, a timber tree, or can be obtained as a synthetic product. Camphor is readily absorbed in the skin and produces a warming sensation when vigorously applied, or a cooling sensation when gently applied. It can also produce a local analgesic effect. Like menthol, camphor also has counterirritant properties. In some embodiments, the topical analgesic spray concentrate comprises from 0.2 wt. % to 11 wt. % camphor, from 1 wt. % to 11 wt. % camphor, 3 wt. % to 11 wt. % camphor, from 5 wt. % to 11 wt. % camphor, or from 8 wt. to 11 wt. % camphor. In certain embodiments, the spray concentrate comprises 5.5 wt. % camphor. In other embodiments, the topical analgesic spray composition comprises from 0.5 wt. % to 3 wt. %, from 1 wt. % to 3 wt. %, from 0.04 wt. % to 2.2 wt. % camphor, from 0.2 wt. % to 2.2 wt. % camphor, from 0.6 wt. % to 2.2 wt. % camphor, from 1 wt. % to 2.2 wt. % camphor, or from 1.6 wt. % to 2.2 wt. % camphor. In certain embodiments, the topical analgesic spray composition comprises 1.1 wt. % or 2.2 wt. % camphor.

As described above, the topical analgesic spray concentrates and topical analgesic spray compositions of the present disclosure may contain a combination of menthol and camphor as active ingredients. It should also be recognized that menthol and camphor combined at certain concentrations or ratios may result in a eutectic mixture. A eutectic mixture is a mixture containing two or more components that has a lower melting point than the separate melting points of its individual constituents. Although menthol and camphor are individually solid at room temperature, the combination of menthol and camphor is known to form liquid, eutectic mixtures at certain ratios.

Certain combinations of concentrations of menthol and camphor may be particularly suitable for the topical analgesic spray concentrates and topical analgesic spray compositions as described herein, including but not limited to, for example, concentrations that result in a eutectic mixture of menthol and camphor. These eutectic mixtures may be more readily formulated than the corresponding non-eutectic compositions, as the liquid phase of the eutectic mixture promotes uniform distribution of the active ingredients throughout the formulation and facilitates absorption into the skin upon application for a rapid pain relieving effect.

In some embodiments, the topical analgesic spray concentrate comprises 16 wt. % menthol and 11 wt. % camphor, 16 wt. % menthol and 5.5 wt. % camphor, 8 wt. % menthol and 2 wt. % camphor, 7 wt. % menthol and 3 wt. % camphor, 6 wt. % menthol and 4 wt. % camphor, or 5 wt. % menthol and 5 wt. % camphor. In certain embodiments, the topical analgesic spray concentrate comprises 16 wt. % menthol and 5.5 wt. % camphor. In other embodiments, the topical analgesic spray composition comprises 3.2 wt. % menthol and 2.2. wt. % camphor, 3.2 wt. % menthol and 1.1 wt. % camphor, 1.6 wt. % menthol and 0.4 wt. % camphor, 1.4 wt. % menthol and 0.6 wt. % camphor, 1.2 wt. % menthol and 0.8 wt. % camphor, or 1 wt. % menthol and 1 wt. camphor. In certain other embodiments, the topical analgesic spray composition comprises 3.2 wt. % menthol and 1.1 wt. % camphor.

It should further be recognized that the topical analgesic spray concentrates and topical analgesic spray compositions may be characterized by the combined concentration of the two active ingredients or as a concentration of a single menthol-camphor mixture. For example, in some embodiments, a topical analgesic spray concentrate may include from 5 to 35 wt. %, from 15 to 35 wt. %, or from 20 to 35 wt. % menthol-camphor mixture. In some embodiments, a topical analgesic spray concentrate may include more than 5 wt. %, more than 10 wt. %, more than 15 wt. %, more than 20 wt. %, more than 25 wt. %, or more than 30 wt. % menthol-camphor mixture. In some embodiments, a topical analgesic spray concentrate may include less than 35 wt. %, less than 30 wt. %, less than 25 wt. %, less than 20 wt. %, less than 15 wt. %, or less than 10 wt. % menthol-camphor mixture. In some embodiments, the topical analgesic spray concentrate of the present disclosure comprises menthol and camphor, wherein the combined concentration of menthol and camphor is at least 10 wt. %, at least 12 wt. %, at least 15 wt. % at least 17 wt. %, at least 20 wt. % or at least 21 wt. %. In certain embodiments, the topical analgesic spray concentrate has a combined concentration of menthol and camphor of at least 10 wt. % or at least 20 wt. %. In certain embodiments, the topical analgesic spray concentrate has a combined concentration of menthol and camphor of 10 wt. %, 21.5 wt. %, or 27 wt. %. If the menthol-camphor mixture is much greater than 35 wt. %, the mixture may have difficulties mixing into solution with the solvent and other components of topical analgesic spray concentrates provided herein.

In some embodiments, a topical analgesic spray composition may include from 1 to 7 wt. %, from 3 to 7 wt. %, or from 4 to 7 wt. % menthol-camphor mixture. In some embodiments, a topical analgesic spray in some embodiments, a topical analgesic spray concentrate may include from 5 to 35 wt. %, from 15 to 35 wt. %, or from 20 to 35 wt. % menthol-camphor mixture. In some embodiments, a topical analgesic spray composition may include more than 5 wt. %, more than 10 wt. %, more than 15 wt. %, more than 20 wt. %, more than 25 wt. %, or more than 30 wt. % menthol-camphor mixture. In some embodiments, a topical analgesic spray composition may include less than 35 wt. %, less than 30 wt. %, less than 25 wt. %, less than 20 wt. %, less than 15 wt. %, or less than 10 wt. % menthol-camphor mixture. In some embodiments, a topical analgesic spray composition may include more than 1 wt. %, more than 2 wt. %, more than 3 wt. %, more than 4 wt. %, more than 5 wt. %, or more than 6 wt. % menthol-camphor mixture. In some embodiments, a topical analgesic spray composition may include less than 7 wt. %, less than 6 wt. %, less than 5 wt. %, less than 4 wt. %, less than 3 wt. %, or less than 2 wt. % menthol-camphor mixture. In other embodiments, the topical analgesic spray composition comprises menthol and camphor, wherein the combined concentration of menthol and camphor is at least 2 wt. %, at least 2.4 wt. %, at least 3 wt. %, at least 3.4 wt. %, at least 4 wt. %, or at least 4.2 wt. %. In certain embodiments, the topical analgesic spray composition has a combined concentration of menthol and camphor of 2 wt. %, 4.3 wt. %, or 5.4 wt. %.

It should be recognized that the solvents, propellants and other excipients described herein may not only be useful for delivery of a wide range of concentrations of menthol and camphor, including menthol and camphor in high concentrations and/or in eutectic mixtures, but also the delivery of additional active ingredients. Numerous different active ingredients may be used in the topical analgesic compositions provided herein. In addition, histamine dihydrochloride, methyl salicylate, methyl nicotinate, and/or capsaicin may also be used in some embodiments. When applied topically, these additional active ingredients can temporarily reduce the pain associated with the musculoskeletal system. Topical analgesic compositions comprising menthol and camphor as provided herein may further include histamine dihydrochloride, methyl salicylate, methyl nicotinate, capsaicin, or any combination thereof.

For example, in some embodiments, the topical analgesic spray concentrate comprises from 0.025 wt. % to 1 wt. %, 0.025 wt. % to 0.750 wt. %, 0.025 wt. % to 0.500 wt. %, or 0.025 wt. % to 0.250 wt. % histamine dihydrochloride. In other embodiments, the topical analgesic spray composition comprises from 0.005 wt. % to 0.200 wt. %, 0.005 wt. % to 0.150 wt. %, 0.005 wt. % to 0.100 wt. %, or 0.005 wt. % to 0.050 wt. % histamine dihydrochloride. In certain embodiments, the topical analgesic spray composition comprises from 0.005 wt. % to 0.050 wt. % histamine dihydrochloride.

### Solvent

The topical analgesic spray concentrates and topical analgesic spray compositions also contain solvent. More specifically, the topical analgesic spray concentrate and topical analgesic spray composition of the present disclosure contain a highly evaporative alcoholic solvent that stabilizes and solubilizes menthol and camphor in the formulation, enables uniform aerosolization of the two active ingredients, and also volatilizes on the skin rapidly to provide a quick-drying, no-drip application of the active ingredients to the site of muscle and joint ache or pain.

In some embodiments, the topical analgesic spray concentrate and topical analgesic spray composition comprise ethanol. In certain embodiments, the topical analgesic spray concentrate and topical analgesic spray composition comprise denatured ethanol. In some embodiments, the analgesic spray concentrate comprises 50 wt. % or more ethanol, 60 wt. % or more ethanol, or 70 wt. % or more ethanol. In other embodiments, the analgesic spray concentrate comprises from 40 wt. % to 80 wt. %, from 50 wt. % to 80 wt. %, from 50 wt. % to 70 wt. %, from 60 wt. % to 80 wt. %, or from 60 wt. % to 70 wt. % ethanol. In yet other embodiments, the topical analgesic spray composition comprises 10 wt. % or more ethanol, 12 wt. % or more ethanol, or 15 wt. % or more ethanol. In still other embodiments, the analgesic spray composition comprises from 8 wt. % to 16 wt. %, from 10 wt. % to 15 wt. %, from 12 wt. % to 16 wt. %, or from 12 wt. % to 15 wt. % ethanol.

Water is not included in the topical analgesic spray concentrates and compositions of the present disclosure. Water is omitted in the topical analgesic spray concentrates and compositions as described herein in order to minimize both the drying time and reduce the occurrence of droplet formation when applied to the skin (i.e., dry, no-drip formulation). In some embodiments of the foregoing, the topical analgesic spray concentrate and topical analgesic spray composition do not contain water.

### Propellants

As described above, an important aspect of the analgesics spray compositions containing high concentrations of menthol and camphor is the high proportion of propellants admixed with the topical analgesic spray concentrate to provide the topical analgesic spray composition in its administrable form. As noted above, the propellants constitute the majority component in the overall topical analgesic spray composition. In particular, the spray compositions of the present disclosure combine the spray formulation with a large proportion of a propellant mixture containing a majority of isopentane as a primary propellant as well as secondary propellants isobutane and propane in a lower concentration. The relatively high proportion of propellants to the spray concentrate is important to facilitate the delivery of menthol and camphor in aerosol form, despite the oiliness and high concentrations of said actives. Due to the large fraction of propellants, and especially of isopentane, the topical analgesic spray composition can be administered as an aerosol while the menthol and camphor within the aerosol droplets remain evenly distributed and solubilized.

In some embodiments, the analgesic spray composition comprises 50 wt. % or more, 60 wt. % or more, 70 wt. % or more, or 75 wt. % or more of one or more propellants. In certain embodiments, the topical analgesic spray composition comprises between 50 wt. % and 90 wt. % of one or more propellants. In other embodiments the analgesic spray composition comprises 80 wt. % of one or more propellants.

Suitable propellants may include, for example, volatile hydrocarbon propellants, such as propane, isopentane, isobutane, etc. In some embodiments, the analgesic spray composition comprises one or more propellants, wherein the one or more propellants are hydrocarbon propellants. In certain embodiments, the one or more propellants are selected from the group consisting of propane, isopentane, isobutane, and any mixtures thereof. In some embodiments, the topical analgesic spray composition comprises isopentane. In other embodiments, the topical analgesic spray composition comprises a mixture of propane and isobutane. In other embodiments, the one or more propellants comprises propane, isopentane or isobutane, or any combinations thereof.

It should be noted that particular amounts or relative quantities of the one or more propellants may be especially useful for preparing the analgesic spray compositions as described herein and providing the desired sensory properties of minimal drying time, minimal drip, and non-greasy feel. For example, at room temperature, isopentane is a volatile liquid, which can serve as a solvent and/or propellant under various conditions. In the topical analgesic spray compositions of the present disclosure, isopentane is utilized as the primary propellant. The majority fraction of isopentane provided in the topical spray composition is such that a balance is achieved between solubilizing and aerosolizing camphor and menthol. In some embodiments, the topical analgesic spray composition comprises more than 50 wt. %, more than 55 wt. %, more than 60 wt. %, more than 65 wt. % or more than 70 wt. % isopentane. In other embodiments, the topical analgesic spray composition comprises less than 90 wt. %, less than 85 wt. % or less than 80 wt. % isopentane. In other embodiments, the topical analgesic spray composition comprises from 50 wt. % to 90 wt. %, from 50 wt. % to 80 wt. %, from 50 wt. % to 70 wt. %, or from 50 wt. % to 60 wt. % isopentane.

In still other embodiments, the topical analgesic spray composition comprises more than 10 wt. %, more than 15 wt. %, more than 20 wt. %, or more than 25 wt. % of a mixture of isobutane and propane. In other embodiments, the topical analgesic composition comprises less than 40 wt. %, less than 35 wt. %, or less than 30 wt. % of a mixture of isobutane and propane. In some embodiments, the topical analgesic spray composition comprises from 10 wt. % to 30 wt. %, from 10 wt. % to 25 wt. %, from 15 wt. % to 30 wt. %, or from 20 wt. % to 30 wt. % of a mixture of isobutane and propane. In certain embodiments, the topical analgesic spray composition comprises at least 50 wt. % isopentane and at least 20 wt. % of a mixture of isobutane and propane. In certain embodiments, the topical analgesic spray composition comprises from 50 wt. % to 70 wt. % isopentane and from 10 wt. % to 30 wt. % of a mixture of isobutane and propane.

Existing spray formulations of menthol and/or camphor typically utilize isobutane as the sole propellant for aerosol systems, or other individual hydrocarbon propellants having comparably high vapor pressures, if a propellant is used at all. In the present disclosure, the use of isopentane as a primary propellant in a ternary propellant system has been unexpectedly found to provide an additional sensory benefit in the topical analgesic spray compositions described herein. It has been surprisingly discovered that the use of isopentane, particularly in a system of isopentane, isobutane and propane, as the dominant propellant in the topical analgesic spray compositions of the present disclosure produces an augmented cooling effect.

Without being bound by theory, it is believed that this enhanced cooling effect is due to the vapor pressure of isopentane, which is relatively lower compared to those of isobutane and propane, but higher than that of ethanol solvent. Inside the aerosol spray dispenser (e.g., a canister or bottle), the topical analgesic spray composition is kept under high pressure, such that the propellants are liquefied within. As the topical analgesic spray composition is dispensed and applied to the skin, the secondary propellants isobutane and propane evaporate almost immediately upon exiting the spray canister due to their extremely high volatility and change in environment from the pressurized canister to atmospheric pressure. The evaporation of isobutane and propane provides an evenly distributed aerosol of the spray formulation. In contrast, isopentane is significantly less volatile than isobutane and propane. Consequently, a small but appreciable amount of isopentane reaches the surface of the skin. Upon reaching the surface of the skin, isopentane subsequently evaporates. The evaporation of the isopentane provides the sensation of cooling as the gaseous isopentane conducts heat away from the consumer's skin, via an evaporative cooling effect.

### Film-Forming Agent

In some embodiments, the concentrates and compositions herein comprise a film-forming agent. Film-forming agents are commonly employed in topical cosmetics and medications to provide smooth skin feel to the consumer during application. In certain embodiments, the film-forming agent is a film-forming agent suitable for aerosolization. By virtue of the large quantity of the propellants in the topical analgesic spray composition relative to the spray concentrate, the forcible, high-velocity expulsion of the topical analgesic spray composition from a pressurized dispenser has the potential to destabilize the active ingredients during application. The inclusion of a film-forming agent in the topical analgesic spray concentrates and topical analgesic spray compositions herein adds to the cohesion of menthol and camphor in the aerosol spray, and thus contributes to the stability of the formulation during application.

In some embodiments, the topical analgesic spray concentrate comprises the topical analgesic spray composition comprises from 0.05 wt. % to 1 wt. %, from 0.05 wt. % to 0.5 wt. %, from 0.05 wt. % to 0.25 wt. %, or from 0.05 wt. % to 0.15 wt. % film-forming agent. In certain embodiments, the topical analgesic spray concentrate comprises from 0.05 wt. % to 1.0 wt. % film-forming agent. In certain embodiments, the topical analgesic spray concentrate comprises 0.1 wt. % film-forming agent. In other embodiments, the topical analgesic spray composition comprises from 0.01 wt. % to 0.2 wt. %, from 0.01 wt. % to 0.1 wt. %, from 0.01 wt. % to 0.05 wt. %, or from 0.01 wt. % to 0.03 wt. % film-forming agent. In certain embodiments, the topical analgesic spray composition comprises from 0.01 wt. % to 0.2 wt. % film-forming agent. In certain embodiments, the topical analgesic spray composition comprises 0.02 wt. % film-forming agent.

In still other embodiments, the topical analgesic spray concentrate and topical analgesic spray composition comprise a film-forming agent, wherein the film-forming agent is a copolymer. In certain embodiments, the film-forming agent is a terpolymer of vinylpyrrolidone, vinyl caprolactum and dimethylaminoethyl methacrylate, such as Advantage™ LC-A.

In addition to the benefit of stability, it has been surprisingly found that the use of particular film-forming agents, such as a terpolymer of vinylpyrrolidone, vinyl caprolactum and dimethylaminoethyl methacrylate, facilitates the localization of the active ingredients onto the skin following application and contributes to the durability of the actives for long-lasting therapeutic effect. Surprisingly, the film-forming agent as described herein contributes to the in situ formation of microscale encapsulating matrices that retain the menthol and camphor in small reservoirs/deposits upon contacting and drying on the skin. In contrast, typical topical compositions employ pre-formulated microbeads or film-forming agents that form vesicles containing active ingredients in the formulation prior to administration, which may interfere with aerosolization. Moreover, the film-forming agent provides a measure of water repellence, thus further prolonging the effect of pain relief.

### Drying Properties of the Topical Analgesic Spray Composition

As described herein, the topical analgesic spray concentrates utilize highly evaporative solvent, which when combined with a large proportion of propellant in the topical analgesic spray composition, allow for rapid drying of the formulation on the consumer's skin. The quick-drying properties of the spray concentrates and spray compositions as described herein may be characterized by quantitative measures, for example, gravimetric evaluation of drying rate or drying speed.

Gravimetric evaluation of drying rate for the topical analgesic spray compositions as described herein may be carried out by spraying a fixed quantity of the composition (e.g., 1 gram) onto a container of known mass (e.g., tared weigh boat or inert similar receptacle) on a measuring scale, and recording the change between the initial and final masses over a specified period of time (such as five minutes), under specified temperature and humidity conditions (such as 75° F.±10° F. and 30%±10% relative humidity). The drying rate (g/min) may then be calculated by dividing the observed loss of mass (g) by the time elapsed (min). The calculated drying rate may be calculated from a single measurement or the average of two or more separate drying rate measurements conducted at the same temperature and humidity conditions.

The temperature and the relative humidity may influence the observed drying rate. In some embodiments, the drying rate is determined at ambient temperature and humidity. In other embodiments, the drying rate is determined at room temperature. In certain embodiments, the drying rate is determined at a temperature of at least about 65° F., at least about 68° F., at least about 70° F., at least about 72° F., or at least about 75° F. In other embodiments, the drying rate is determined at a temperature of less than or equal to about 85° F., less than or equal to about 82° F., less than or equal to about 80° F., less than or equal to about 77° F., or less than or equal to about 75° F. In certain embodiments, the drying rate is determined at a temperature of between about 68° F. and about 77° F. (20° C. and about 25° C.). In still certain other embodiments, the drying rate is determined at a temperature of about 75° F.±10° F.

In yet other embodiments, which may be combined with any of the preceding embodiments, the drying rate is determined at a relative humidity of at least about 20%, at least about 30%, at least about 40%, at least about 50%, or at least about 60%. In other embodiments, the drying rate is determined at a relative humidity of less than or equal to about 80%, less than or equal to about 70%, or less than or equal to about 60%. In certain embodiments, the drying rate is determined at a relative humidity of between about 30% and about 70%. In certain embodiments, the drying rate is determined at a relative humidity of about 30%±10%.

In some embodiments, the topical analgesic spray composition has a drying rate of at least about 0.03 g/min, at least about 0.04 g/min, at least about 0.05 g/min, at least about 0.06 g/min, at least about 0.07 g/min, at least about 0.075 g/min, at least about 0.08 g/min, at least about 0.09 g/min, at least about 0.1 g/min, at least about 0.11 g/min or at least about 0.12 g/min as determined by gravimetric evaluation as described herein. In certain embodiments, the topical analgesic spray composition has a drying rate of at least about 0.03 g/min, at least about 0.04 g/min, at least about 0.05 g/min, at least about 0.06 g/min, at least about 0.07 g/min, at least about 0.075 g/min, at least about 0.08 g/min, at least about 0.09 g/min, at least about 0.1 g/min, at least about 0.11 g/min or at least about 0.12 g/min as determined by gravimetric evaluation at a temperature of about 75° F.±10° F. and a relative humidity of about 30%±10%.

As the propellants provided in the topical analgesic spray compositions are expected to have dissipated by the time the spray concentrate reaches the desired surface (that is, skin in the case of application or a weigh boat in the case of gravimetric evaluation), the topical analgesic spray concentrates may also be described by the same drying rates. In some variations, the topical analgesic spray concentrate has a drying rate of at least about 0.03 g/min, at least about 0.04 g/min, at least about 0.05 g/min, at least about 0.06 g/min, at least about 0.07 g/min, at least about 0.075 g/min, at least about 0.08 g/min, at least about 0.09 g/min, at least about 0.1 g/min, at least about 0.11 g/min or at least about 0.12 g/min as determined by gravimetric evaluation as described herein. In certain embodiments, the topical analgesic spray concentrate has a drying rate of at least about 0.03 g/min, at least about 0.04 g/min, at least about 0.05 g/min, at least about 0.06 g/min, at least about 0.07 g/min, at least about 0.075 g/min, at least about 0.08 g/min, at least about 0.09 g/min, at least about 0.1 g/min, at least about 0.11 g/min or at least about 0.12 g/min as determined by gravimetric evaluation at a temperature of about 75° F.±10° F. and a relative humidity of about 30%±10%.

### Additional Ingredients

In addition to the above, the topical analgesic spray concentrate and topical analgesic spray composition of the present disclosure may include further ingredients to modify the aesthetic properties of the formulations. The additional ingredients may be incorporated into the topical analgesic spray concentrates and compositions as described above without detracting quick-drying properties or skinfeel.

In other embodiments, the topical analgesic spray concentrate and topical analgesic spray composition comprise fragrance. Although menthol and camphor have their own distinct scents that contribute to the overall aroma of the topical analgesic spray concentrate and topical analgesic spray composition, additional fragrance may be included in to modify the olfactive properties of the spray. For example, a fragrance blend may include one or more fragrances including, but not limited to, sage, bergamot, spearmint, lemon, rose, jasmine, lavender, cedar wood, amber, musk, and/or eucalyptus. In some embodiments, a fragrance blend may provide a pleasant fragrance as a mask, and/or to complement the natural scent of menthol and camphor. For example, a fragrance blend may provide a mint effect with soothing qualities and botanical facets. In some embodiments, the topical analgesic spray concentrate comprises from 0.5 wt. % to 2 wt. %, from 0.5 wt. % to 1.5 wt. %, or from 0.5 wt. % to 1 wt. % fragrance. In other embodiments, the topical analgesic spray composition comprises from 0.1 wt. % to 0.4 wt. %, from 0.1 wt. % to 0.3 wt. %, or from 0.1 wt. % to 0.2 wt. % fragrance.

Propanediol may also be incorporated into the topical analgesic spray concentrates and spray compositions of the present disclosure. Propanediol has varied utility in topical formulations including as a humectant and emollient and may be added to modify skin feel properties of the spray formulations. In some embodiments of the foregoing, the topical analgesic spray concentrate and topical analgesic spray composition comprise propanediol. In certain embodiments, the topical analgesic spray concentrate comprises 0.1 wt. % to 2 wt. %, from 0.5 wt. % to 1.5 wt. %, or from 0.5 to 1.0 wt. % propanediol. In certain embodiments, the topical analgesic spray concentrate comprises 1 wt. % propanediol. In yet other embodiments, the topical analgesic spray composition comprises 0.02 wt. % to 0.4 wt. %, from 0.1 wt. % to 0.3 wt. %, or from 0.1 to 0.2 wt. % propanediol. In certain embodiments, the topical analgesic spray composition comprises 0.2 wt. % propanediol.

Similar to propanediol, dimethyl isosorbide is another common excipient for topical applications, which is utilized as a solvent and/or thinning agent to reduce viscosity, and which can be incorporated into the topical analgesic spray concentrate and spray compositions provided herein. In some embodiments, the topical analgesic spray composition comprises dimethyl isosorbide. In some embodiments, the topical analgesic spray concentrate comprises from 0.1 wt. % to 1 wt. %, from 0.2 wt. % to 0.8 wt. %, or from 0.3 wt. % to 0.7 wt. % dimethyl isosorbide. In certain embodiments, the topical analgesic spray concentrate comprises 0.5 wt. % dimethyl isosorbide. In other embodiments, the topical analgesic spray composition comprises from 0.02 wt. % to 0.2 wt. %, from 0.04 wt. % to 0.16 wt. %, or from 0.06 wt. % to 0.14 wt. % dimethyl isosorbide. In certain embodiments, the topical analgesic spray composition comprises 0.1 wt. % dimethyl isosorbide.

As described in detail below, topical analgesics and/or topical analgesic compositions provided herein may comprise an organoleptic composition. An organoleptic composition according to embodiments provided herein may include cooling and warming sensates, an essential oil mixture, linseed oil, and optionally also further excipients (such as vitamin E oil, surfactants, penetration enhancers).

### Organoleptic Composition

Mentholated and camphorated formulations are commonly perceived as having a strong medicinal odor. The organoleptic compositions as described herein may be incorporated into the topical analgesic spray formulations to add to the sensation of long-lasting pain-relieving effect and/or to provide pleasant fragrance as a mask and/or complement to the natural scent of menthol and camphor. The organoleptic compositions of the present disclosure provide these sensorial effects without detracting from the quick-drying, non-greasy skin feel properties of the spray formulations.

Disclosed herein are organoleptic compositions that can include cooling and warming sensates, an essential oil mixture, linseed oil, and optionally also further excipients (such as vitamin E oil, surfactants, penetration enhancers) for inclusion in the topical analgesic spray concentrates and compositions containing high concentrations of menthol and camphor provided herein. In some embodiments of the present disclosure, the topical analgesic spray concentrates and topical analgesic spray compositions comprise an organoleptic composition as described herein. Components of an organoleptic composition described in detail below may be included in a topical analgesic spray concentrate and/or in a topical analgesic spray composition in addition to, or in lieu of, one or more components described above with reference the topical analgesic spray concentrate and/or the topical analgesic spray composition. For example, organoleptic compositions provided herein may include linseed oil, and topical analgesic spray concentrates and/or topical analgesic spray compositions provided herein may include linseed oil. Thus, topical analgesic spray concentrates and/or topical analgesic spray compositions provided herein may comprise no linseed oil, one dose of linseed oil (i.e., either that disclosed with reference to a topical analgesic and/or a topical analgesic composition or that disclosed with reference to an organoleptic composition), or two doses of linseed oil (i.e., that disclosed with reference to both the topical analgesics and/or topical analgesic compositions and the organoleptic compositions).

In some embodiments, the organoleptic composition comprises one or more sensates. In certain embodiments wherein the organoleptic composition comprises one or more sensates, the one or more sensates are selected from the group consisting of cooling sensates, warming sensates, and any combinations or mixtures thereof. Suitable cooling and warming sensates may include but are not limited to menthol and menthol derivatives (e.g., isomenthol, neomenthol, neoisomenthol, menthoglycol para-menthoxy-3,8-propanediol, isopulegol), capsaicin, other capsaicinoids (e.g., dihydrocapsaicin, nordihydrocapsaicin, homocapsaicin, and homodihydrocapsaicin), eucalyptol, cinnamaldehyde, vanilloid derivatives such as vanillyl alcohol alkyl ethers (e.g., vanillyl alcohol n-butyl ether, vanillyl alcohol n-propyl ether, vanillyl alcohol isopropyl ether, vanillyl alcohol isobutyl ether, vanillyl alcohol n-amino ether, vanillyl alcohol n-hexyl ether, vanillyl amyl ether, vanillyl alcohol methyl ether, vanillyl alcohol ethyl ether, vanillyl isoamyl ether), gingerol, zingerone, shogaol, piperine, icilin, and any combinations thereof. In some embodiments, the organoleptic composition comprises menthoxypropanediol (Coolact® 10), isopulegol (Coolact® P), or icilin, or a combination thereof, as cooling sensates. In other embodiments, the organoleptic composition comprises vanillyl butyl ether (Hotact® VBE), cinnamaldehyde, or piperine, or a combination thereof, as warming sensates.

Menthoxypropanediol is a sensate and synthetic derivative of menthol that can provide a cooling sensation when applied to the skin. The compound acts as a cooling agent by stimulating the receptors at the nerve endings of the skin where applied to produce a cooling sensation. Menthoxypropanediol can also be used as a fragrance or a masking ingredient in some formulations. Too much menthoxypropanediol can cause irritation and even chemical burning. Too little menthoxypropanediol in a topical analgesic formulation may render the formulation less effective at producing a cooling sensation. In some embodiments, the topical analgesic spray concentrate comprising the organoleptic composition provided herein includes from 2 to 40 wt. %, from 5 to 30 wt. %, or from 10 to 20 wt. % menthoxypropanediol. In some embodiments, an organoleptic composition comprises less than 40 wt. %, less than 35 wt. %, less than 30 wt. %, less than 25 wt. %, less than 20 wt. %, less than 15 wt. %, less than 10 wt. %, or less than 5 wt. % menthoxypropanediol. In some embodiments, an organoleptic composition comprises more than 2 wt. %, more than 5 wt. %, more than 10 wt. %, more than 15 wt. %, more than 20 wt. %, more than 25 wt. %, more than 30 wt. %, or more than 35 wt. % menthoxypropanediol.

Isopulegol is a sensate that is a chemical precursor to menthol. It is a terpene found in cannabis and known for having a minty odor. However, isopulegol also has anxiolytic, gastroprotective, and anticonvulsive properties. When used in the topical analgesic compositions provided herein, isopulegol can be used as a sensate that provides a cooling effect to the skin. It can function as a sensate by directly stimulating the receptors at the nerve endings of the skin to produce a cooling sensation. Specifically, isopulegol can provide a similar cooling effect as menthol, but without the odor of menthol. Topical analgesic compositions having too much isopulegol can be irritating to the skin. However, topical analgesic compositions having too little isopulegol may render the formulation less effective at providing the desired cooling effect. In some embodiments, a topical analgesic spray concentrate and/or a topical analgesic spray composition comprising the organoleptic composition provide herein includes from 2 to 40 wt. %, from 5 to 30 wt. %, or from 10 to 20 wt. % isopulegol. In some embodiments, an organoleptic composition comprises less than 40 wt. %, less than 35 wt. %, less than 30 wt. %, less than 25 wt. %, less than 20 wt. %, less than 15 wt. %, less than 10 wt. %, or less than 5 wt. % isopulegol. In some embodiments, an organoleptic composition comprises more than 2 wt. %, more than 5 wt. %, more than 10 wt. %, more than 15 wt. %, more than 20 wt. %, more than 25 wt. %, more than 30 wt. %, or more than 35 wt. % isopulegol.

Vanillyl butyl ether is a sensate that provides a warming effect when applied to the skin. The warming effect of vanillyl butyl ether can occur immediately upon application, building rapidly within the first five minutes and lasting up to two hours. Compared to active ingredients that can produce a warming effect (e.g., capsaicin or capsicum extract), vanillyl butyl ether can be less irritating. That said, topical analgesic compositions comprising too much vanillyl butyl ether can still cause skin irritation and/or burning. Topical analgesic compositions comprising too little vanillyl butyl ether may render the formulation less effective at providing the desired warming effect. In some embodiments, an organoleptic composition provided herein includes from 0.1 to 15 wt. %, from 1 to 10 wt. %, or from 2 to 5 wt. % vanillyl butyl ether. In some embodiments, an organoleptic composition provide herein includes less than 15 wt. %, less than 10 wt. %, less than 8 wt. %, less than 5 wt. %, less than 3 wt. %, less than 1 wt. %, or less than 0.5 wt. % vanillyl butyl ether. In some embodiments, an organoleptic composition provide herein includes more than 0.1 wt. %, more than 0.5 wt. % more than 1 wt. %, more than 3 wt. %, less than 5 wt. %, more than 8 wt. % or more than 10 wt. % vanillyl butyl ether.

In addition to individual chemical compounds that may provide cooling or warming sensation, the organoleptic composition may also comprise naturally derived extracts, roots, or resins containing one or more sensates. For example, in some embodiments, the organoleptic composition may comprise chili pepper (Capsicum frutescens) resin, ginger root extract and cinnamon cassia bark extract, or any combination thereof. Combinations of particular naturally derived extracts, roots, or resins may also be known and referred to by known trade name(s). In certain embodiments, the organoleptic composition comprises Phytol™ Heat (a combination of chili pepper (Capsicum frutescens) resin, ginger root extract and cinnamon cassia bark extract.

In some embodiments of the organoleptic composition, a complementary cooling effect to supplement the effects of camphor and menthol is desired and particular combinations of certain cooling sensates may be incorporated into the organoleptic composition, and ultimately the topical analgesic spray concentrates and topical analgesic spray compositions, in order to achieve the desired cooling sensation. In some embodiments, the one or more sensates are selected from the group consisting of menthoxypropanediol, isopulegol, and icilin, and any combinations thereof.

In other embodiments of the organoleptic composition, a complementary warming sensation is desired to supplement the effects of camphor and menthol. Similar combinations of warming sensates may be incorporated into the organoleptic composition, and consequently also, the topical analgesic spray concentrates and topical analgesic spray compositions, in order to achieve the desired warming sensation. In some embodiments, the one or more sensates are selected from the group consisting of cinnamaldehyde, piperine, and vanillyl butyl ether, and any combinations thereof.

A combined cooling and warming sensation may be desired in some topical analgesic spray concentrates and topical analgesic compositions. To achieve a combined cooling and warming sensation, combinations of certain cooling and warming sensates may be incorporated into the organoleptic composition to achieve the mixed cooling/warming effect. In certain embodiments, the organoleptic composition comprises one or more sensates selected from the group consisting of menthoxypropanediol, isopulegol, vanillyl butyl ether, a combination of chili pepper (Capsicum frutescens) resin, ginger root extract and Cinnamon cassia bark extract, and any combinations thereof. In some embodiments, the one or more sensates are selected from the group consisting of menthoxypropanediol, isopulegol, and vanillyl butyl ether, and any combinations thereof.

Essential oils may also be included in the organoleptic compositions as described herein to complement the effect of the menthol, camphor and aforementioned sensates on hot and cold receptors in the skin and/or to imbue an overall pleasant fragrance to the topical analgesic compositions. In addition to their sensory attributes, the essential oils utilized in the organoleptic compositions, topical analgesic spray concentrates and topical analgesic spray compositions described herein may further provide anti-inflammatory, anti-oxidant and/or antinociceptive effects on the skin. In some embodiments, the organoleptic composition may comprise an essential oil mixture comprising one or more essential oils. In some embodiments, the organoleptic composition comprises an essential oil mixture comprising one or more essential oils selected from the group consisting of peppermint (Mentha piperita) oil, eucalyptus (Eucalyptus globulus) oil, rosemary (Rosmarinus officinalis) oil, Tunisian rosemary (Rosmarinus officinalis) oil, Idaho rosemary (Rosmarinus officinalis) oil, clove (Eugenia caryophyllata) oil, Spanish marjoram (Thymus mastichina) oil, sweet marjoram (Organum majorana) oil frankincense (Olibanum or Boswellia carterii) oil, clove (Syzygium aromaticum) oil, Ceylon cinnamon (Cinnamomum verum or zeylanicum) oil, cardamom (Elettaria cardamomum) oil, Guatemalan cardamom (Elettaria cardamomum) oil, black pepper (Piper nigrum) oil, bay leaf (or bay laurel or Laurus nobilis) oil, cassia (Cinnamomum cassia) oil, ginger (Zingiber officinale) oil, Chinese ginger (Zingiber officinale) oil, lemongrass Cochin (Cymbopogon citratus) oil, fennel (Foeniculum vulgare) oil, basil (Ocimum basilicum) oil, spearmint (Mentha spicata or cardiaca) oil, Roman chamomile (Anthemis nobilis of Chamaemelum nobile) oil, sage (Salvia officinalis L.) oil, Spanish sage (Salvia lavandulaefolia) oil, clary sage (Salvia sclarea) oil, Bulgarian lavender (Lavandula angustifolia or officinalis) oil, and nutmeg (Myristica fragrans) oil.

As described above, combinations of sensates may be prepared to afford a cooling, warming or mixed cooling and warming sensation to the organoleptic composition, and ultimately also to the topical analgesic compositions. Similarly, combinations of essential oils in the essential oil mixture may be prepared to supplement the cooling, warming, or mixed cooling and warming effects of the sensates. Certain combinations or blends of essential oils may be especially suitable for providing cooling sensation and/or warming sensation as desired. For example, in some embodiments wherein a warming sensation is desired, the essential oil mixture comprises clove (Eugenia caryophyllata) oil, Ceylon cinnamon (Cinnamomum verum or zeylanicum) oil, cardamom (Elettaria cardamomum) oil, black pepper (Piper nigrum) oil, bay leaf (or bay laurel or Laurus nobilis) oil, cassia (Cinnamomum cassia) oil, and ginger (Zingiber officinale) oil. In other embodiments wherein a cooling sensation is desired, the essential oil mixture comprises fennel (Foeniculum vulgare) oil, peppermint (Mentha piperita) oil, basil (Ocimum basilicum) oil, spearmint (Mentha spicata or cardiaca) oil, eucalyptus (Eucalyptus globulus) oil, sage (Salvia officinalis L.) oil, and nutmeg (Myristica fragrans) oil. In still other embodiments wherein a mixed cooling and warming sensation is desired, the organoleptic composition comprises an essential oil mixture comprising peppermint (Mentha piperita) oil, eucalyptus (Eucalyptus globulus) oil, rosemary (Rosmarinus officinalis) oil, clove (Eugenia caryophyllata) oil, Spanish marjoram (Thymus mastichina) oil and frankincense (Olibanum or Boswellia carterii) oil. It should be recognized the essential oil mixtures tailored for cooling sensation, warming sensation and mixed cooling and warming sensation can be combined with the respective combinations of sensates for cooling, warming and mixed cooling and warming.

Surprisingly, the essential oil mixture comprising peppermint (Mentha piperita) oil, eucalyptus (Eucalyptus globulus) oil, rosemary (Rosmarinus officinalis) oil, clove (Eugenia caryophyllata) oil, Spanish marjoram (Thymus mastichina) oil and frankincense (Olibanum or Boswellia carterii) oil was discovered to provide a combined cooling and warming sensation as well as effectively mask the smell of menthol and camphor and were compatible with a variety of topical analgesic formulations.

In some embodiments, an organoleptic composition comprises from 2 to 40 wt. %, from 5 to 30 wt. %, or from 10 to 20 wt. % essential oil mixture. In some embodiments, an organoleptic composition comprises less than 40 wt. %, less than 35 wt. %, less than 30 wt. %, less than 25 wt. %, less than 20 wt. %, less than 15 wt. %, less than 10 wt. %, or less than 5 wt. % essential oil mixture. In some embodiments, an organoleptic composition comprises more than 2 wt. %, more than 5 wt. %, more than 10 wt. %, more than 15 wt. %, more than 20 wt. %, more than 25 wt. %, more than 30 wt. %, or more than 35 wt. % essential oil mixture.

It should be recognized that in certain embodiments wherein peppermint oil is included in the essential oil mixture, the peppermint oil may contribute to the total quantity of menthol in the overall topical analgesic formulation. As a result of the contribution of menthol from the peppermint oil, which may depend upon the source of the peppermint oil, the fraction of menthol in the peppermint oil and total concentration of the peppermint oil in the topical analgesic, the quantity of menthol added as an independent ingredient may be adjusted accordingly to achieve the desired concentration.

In certain embodiments wherein the organoleptic composition comprises an essential oil mixture comprising one or more essential oils and vitamin E. Vitamin is a known antioxidant and may be included in the essential oil mixture to prevent oxidation of the individual essential oils for longer shelf life. Vitamin E may be further included in the organoleptic composition as described herein as an antioxidant and emollient, independently of any vitamin E already included essential oil mixture. Vitamin E broadly refers to a group of fat soluble compounds known as tocopherols and tocotrienols, which have free-radical scavenging properties, but as referred to herein may include any individual isomers (alpha, beta, gamma, delta) of tocopherol and/or tocotrienol, or any combinations thereof. In still other embodiments, the organoleptic composition further comprises vitamin E. In some embodiments, an organoleptic composition comprises vitamin E. Vitamin E is a known antioxidant and may be included in the essential oil mixture to prevent oxidation of the individual essential oils for longer shelf life. In some embodiments, an organoleptic composition comprises from 1 to 20 wt. %, from 3 to 15 wt. %, or from 5 to 10 wt. % vitamin E. In some embodiments, an organoleptic composition comprises less than 20 wt. %, less than 15 wt. %, less than 10 wt. %, less than 8 wt. %, less than 5 wt. %, or less than 3 wt. % vitamin E. In some embodiments, an organoleptic composition comprises more than 1 wt. %, more than 3 wt. %, less than 5 wt. %, more than 8 wt. %, more than 10 wt. %, or more than 15 wt. % vitamin E.

Certain relative percentages of the individual essential oils (and vitamin E) may be especially complementary in fragrance and scent. The table below provides one example of a complementary combination of the mixture of essential oils and vitamin E for use with mentholated and camphorated topical formulations. It should be recognized that the individual percentages of each of the essential oils may be varied to provide the desired complementary scent and sensation with respect to the other cooling and/or warming sensates, menthol and camphor. For example, in some embodiments, the percentages of the essential oils shown in the table below may be varied within ±25%. In addition, it should be further recognized that the exemplary essential oil blend shown in the table below is not intended to be limiting and that the essential oils in the organoleptic composition may be substituted to provide greater cooling or warming effect as desired.

| International Nomenclature of Cosmetic | |
|---|---|
| Ingredient | % (with vitamin E) |
| Rosemary (Rosmarinus officinalis) leaf oil | 24.5 |
| Spanish marjoram (Thymus mastichina) flower oil | 24.5 |
| Peppermint (Mentha piperita) oil | 14.5 |
| Eucalyptus (Eucalyptus globulus) leaf oil | 14.5 |
| Clove (Eugenia caryophyllus) oil | 10.0 |
| Frankincense (Boswellia carterii) oil | 10.0 |
| Tocopherol | 2.0 |
| TOTAL | 100.0 |

In some embodiments wherein the topical analgesic spray composition comprises the organoleptic composition provide herein, the topical analgesic spray concentrate and the topical analgesic spray composition comprise an essential oil mixture comprising one or more essential oils and vitamin E. In certain embodiments, the topical analgesic spray concentrate and the topical analgesic spray composition comprise peppermint (Mentha piperita) oil, eucalyptus (Eucalyptus globulus) oil, rosemary (Rosmarinus officinalis) oil, clove (Eugenia caryophyllata) oil, Spanish marjoram (Thymus mastichina) oil and frankincense (Olibanum or Boswellia carterii) oil, and vitamin E.

In some embodiments, the organoleptic composition further comprises linseed oil as an emollient. Linseed oil, also known as flaxseed oil or flax oil, contains a variety of triglycerides, including alpha-linoleic acid, which can help to moisturize skin and enhance skin feel of topical formulations. In some embodiments, an organoleptic composition includes from 0.1 to 15 wt. % or from 1 to 5 wt. % linseed oil. In some embodiments, an organoleptic composition includes less than 15 wt. %, less than 10 wt. %, less than 5 wt. %, less than 4 wt. %, less than 3 wt. %, less than 2 wt. %, less than 1 wt. %, less than 0.8 wt. %, less than 0.5 wt. %, less than 0.3 wt. %, less than 0.1 wt. %, or less than 0.05 wt. % linseed oil. In some embodiments, an organoleptic composition includes more than more than 0.1 wt. %, more than 0.3 wt. %, more than 0.5 wt. %, more than 0.8 wt. %, more than 1 wt. %, more than 2 wt. %, more than 3 wt. %, more than 4 wt. %, more than 5 wt. %, or more than 10 wt. % linseed oil.

In some embodiments, the organoleptic composition comprises one or more excipients, such as surfactants and/or penetration enhancers. In some embodiments the topical analgesic spray composition comprises surfactants. Suitable surfactants may include but are not limited to those derived from functionalization of sorbitan. For example, in some embodiments, the organoleptic composition may comprise sorbitan ester surfactants, ethoxylated sorbitan ester surfactants (polysorbates), or any mixtures thereof. It should be recognized that certain classes of surfactants may be especially useful, including for example, sorbitan ester surfactants, ethoxylated sorbitan ester surfactants (polysorbates), or any mixtures thereof, wherein the ester is moiety is oleate. In certain embodiments, the organoleptic composition comprises surfactants selected from the group consisting of polyethylene glycol sorbitan monooleate (Tween® 80), sorbitan monooleate (Span® 80), sorbitan trioleate (Span® 85), and any combination thereof. In some embodiments, an organoleptic composition may include from 5 to 50 wt. %, from 10 to 45 wt. %, or from 20 to 40 wt. % penetration enhancer. In some embodiments, an organoleptic composition may include more than 5 wt. %, more than 10 wt. %, more than 15 wt. %, more than 20 wt. %, more than 25 wt. %, more than 30 wt. %, more than 35 wt. %, more than 40 wt. %, or more than 45 wt. % of one or more surfactants. In some embodiments, an organoleptic composition may include less than 50 wt. %, less than 45 wt. %, less than 40 wt. %, less than 35 wt. %, less than 30 wt. %, less than 25 wt. %, less than 20 wt. %, less than 15 wt. %, or less than 10 wt. % of one or more surfactants.

In other embodiments, the organoleptic composition comprises a penetration enhancer. In certain embodiments, the penetration enhancer is an alkylene glycol. In still other embodiments, the penetration enhancer is pentylene glycol. In some embodiments, an organoleptic composition may include from 5 to 50 wt. %, from 10 to 45 wt. %, or from 20 to 40 wt. % penetration enhancer. In some embodiments, an organoleptic composition may include more than 5 wt. %, more than 10 wt. %, more than 15 wt. %, more than 20 wt. %, more than 25 wt. %, more than 30 wt. %, more than 35 wt. %, more than 40 wt. %, or more than 45 wt. % penetration enhancer. In some embodiments, an organoleptic composition may include less than 50 wt. %, less than 45 wt. %, less than 40 wt. %, less than 35 wt. %, less than 30 wt. %, less than 25 wt. %, less than 20 wt. %, less than 15 wt. %, or less than 10 wt. % penetration enhancer.

As described above, the organoleptic composition containing sensates, essential oils and linseed oil, and optionally also further excipients, may be incorporated into topical formulations possessing high concentrations of menthol and camphor, including the topical analgesic spray concentrates and topical analgesic spray compositions of the present disclosure.

Although the organoleptic composition are described herein with specific reference to their use in the topical analgesic spray compositions of the present disclosure, it should be recognized that the organoleptic composition as described herein may be tailored for incorporation into different formulation types also having high concentrations of menthol and camphor, including emulsions, gels, etc. It should also be recognized that the organoleptic composition as described herein may be adapted to include varied combinations of the cooling and warming sensates, or varied relative concentrations of the sensates to the essential oil mixture, or even exclude certain or all optional excipients.

### Topical Analgesic Spray Compositions and Concentrates Comprising an Organoleptic Composition

Discussed below are topical analgesic compositions comprising an organoleptic composition as described above. In particular, the compounds/ingredients described below have been introduced with respect to the organoleptic compositions disclosed above and are reiterated below with respect to the topical analgesic composition as a whole. As described in detail above, organoleptic compositions provided herein may be incorporated into topical analgesic compositions to add to the sensation of long-lasting pain-relieving effect, to provide a pleasant fragrance as a mask, and/or to complement the natural scent of menthol and camphor. An organoleptic composition according to embodiments provided herein may include cooling and warming sensates, an essential oil mixture, linseed oil, and optionally also further excipients (such as vitamin E oil, surfactants, penetration enhancers).

In some embodiments, provided herein are topical analgesic spray concentrates and topical analgesic spray compositions comprising an organoleptic composition, wherein the organoleptic composition comprises cooling and warming sensates, an essential oil mixture comprising one or more essential oils and vitamin E, linseed oil, and optionally also further excipients. In certain embodiments, provided herein are topical analgesic spray concentrates and topical analgesic spray compositions comprising an organoleptic composition, wherein the organoleptic composition comprises cooling and warming sensates, an essential oil mixture comprising one or more essential oils and vitamin E, and linseed oil.

In some embodiments, a topical analgesic spray concentrate may include from 1 to 30 wt. %, from 2 to 20 wt. %, or from 3 to 10 wt. % organoleptic composition. In some embodiments, a topical analgesic spray concentrate may include more than 1 wt. %, more than 2 wt. %, more than 3 wt. %, more than 5 wt. %, more than 8 wt. %, more than 10 wt. %, more than 15 wt. %, more than 20 wt. %, or more than 25 wt. % organoleptic composition. In some embodiments, a topical analgesic spray concentrate may include less than 30 wt. %, less than 25 wt. %, less than 20 wt. %, less than 15 wt. %, less than 10 wt. %, less than 8 wt. %, less than 5 wt. %, less than 3 wt. %, or less than 2 wt. % organoleptic composition.

In other embodiments, a topical analgesic spray composition may include from 0.2 to 6 wt. %, from 0.4 to 4 wt. %, or from 0.6 to 2 wt. % organoleptic composition. In some embodiments, a topical analgesic spray composition may include more than 0.2 wt. %, more than 0.4 wt. %, more than 0.6 wt. %, more than 1 wt. %, more than 1.6 wt. %, more than 2 wt. %, more than 3 wt. %, more than 4 wt. %, or more than 5 wt. % organoleptic composition. In some embodiments, a topical analgesic spray composition may include less than 6 wt. %, less than 5 wt. %, less than 4 wt. %, less than 3 wt. %, less than 2 wt. %, less than 1.6 wt. %, less than 1 wt. %, less than 0.6 wt. %, or less than 0.4 wt. % organoleptic composition.

In some embodiments wherein the topical analgesic spray composition comprises the organoleptic composition provide herein, the topical analgesic spray concentrate and topical analgesic spray composition comprise one or more sensates. In some embodiments, the topical analgesic spray concentrate comprises from 5 wt. % to 15 wt. %, from 7 wt. % to 12 wt. %, or from 8 wt. % to 10 wt. % of one or more sensates. In other embodiments, the topical analgesic spray composition comprises from 1 wt. % to 3 wt. %, from 1.4 wt. % to 2.4 wt. %, or from 1.6 wt. % to 2 wt. % of one or more sensates.

In certain embodiments, the topical analgesic spray concentrate and topical analgesic spray composition comprises one or more sensates selected from the group consisting of menthoxypropanediol, isopulegol, and vanillyl butyl ether, and any combination thereof. In certain embodiments, the topical analgesic spray concentrate or topical analgesic spray composition comprises menthoxypropanediol, isopulegol, and vanillyl butyl ether.

As discussed above, menthoxypropanediol is a sensate that can provide a cooling sensation to the skin. In some embodiments, a topical analgesic spray concentrate comprising the organoleptic composition provided herein includes from 0.01 to 10 wt. %, from 0.1 to 5 wt. %, or from 0.5 to 3 wt. % menthoxypropanediol. In some embodiments, a topical analgesic spray composition comprising the organoleptic composition provided herein includes from 0.01 to 5 wt. %, from 0.1 to 3 wt. %, or from 0.5 to 2 wt. % menthoxypropanediol. An example of a commercially-available menthoxypropanediol is Coolact® 10.

As discussed above, isopulegol is a sensate that can provide a cooling effect on the skin. In some embodiments, a topical analgesic composition comprising the organoleptic composition provided herein includes from 0.01 to 10 wt. %, from 0.1 to 5 wt. %, or from 0.5 to 3 wt. % isopulegol. In some embodiments, a topical analgesic spray composition comprising the organoleptic composition provided herein includes from 0.01 to 5 wt. %, from 0.1 to 3 wt. %, or from 0.5 to 2 wt. % isopulegol. An example of a commercially-available isopulegol includes Coolact® P.

As described in detail above, vanillyl butyl ether is a sensate that can provide a warming sensation on the skin. In some embodiments, a topical analgesic spray concentrate comprising the organoleptic composition provided herein includes from 0.01 to 1 wt. %, from 0.05 to 0.5 wt. %, or from 0.05 to 0.1 wt. % vanillyl butyl ether. An example of a commercially-available vanillyl butyl ether is Hotact® VBE. In other embodiments, a topical analgesic spray composition comprising the organoleptic composition provided herein includes from 0.01 to 0.2 wt. %, from 0.01 to 0.1 wt. %, or from 0.01 to 0.02 wt. % vanillyl butyl ether.

In some embodiments, the topical analgesic spray concentrate comprises 5 wt. % menthoxypropanediol, 5 wt. % isopulegol, and 0.05 wt. % vanillyl butyl ether. In other embodiments, the topical analgesic spray composition comprises 1 wt. % menthoxypropanediol, 1 wt. % isopulegol, and 0.01 wt. % vanillyl butyl ether.

In some embodiments, a topical analgesic spray concentrate and spray composition comprising the organoleptic composition provided herein includes an essential oil mixture comprising one or more essential oils and/or vitamin E. Essential oils can provide a more pleasant sensory experience for a user by complementing and/or masking the odors of menthol and/or camphor. Vitamin E, when applied to the skin, can be moisturizing and can help protect the skin from free radical damage. In some embodiments, the essential oil mixture can include one or more of peppermint (Mentha piperita) oil, eucalyptus (Eucalyptus globulus) oil, rosemary (Rosmarinus officinalis) oil, clove (Eugenia caryophyllata) oil, Spanish marjoram (Thymus mastichina) oil and frankincense (Olibanum or Boswellia carterii) oil, and/or vitamin E. In some embodiments, a topical analgesic spray concentrate comprising the organoleptic composition provided herein comprises from 0.01 to 10 wt. %, from 0.1 to 5 wt. %, or from 0.5 to 3 wt. % of an essential oil mixture. In some embodiments, a topical analgesic spray concentrate comprising the organoleptic composition provide herein includes less than 10 wt. %, less than 8 wt. %, less than 5 wt. %, less than 3 wt. %, less than 1 wt. %, or less than 0.5 wt. % of an essential oil mixture. In some embodiments, the topical analgesic includes more than 0.1 wt. %, more than 0.5 wt. % more than 1 wt. %, more than 3 wt. %, more than 5 wt. %, or more than 8 wt. % of an essential oil mixture. In certain embodiments, the topical analgesic spray concentrate comprises from 0.5 wt. to 3 wt. %, from 1 wt. % to 2 wt. %, or from 1 wt. % to 1.5 wt. % of one or more essential oils. In still other embodiments, the topical analgesic spray concentrate comprises 1.1 wt. % of one or more essential oils. In some embodiments, the topical analgesic spray composition comprises from 0.1 wt. % to 0.6 wt. %, from 0.2 wt. % to 0.4 wt. %, or from 0.2 wt. % to 0.3 wt. % of one or more essential oils. In certain embodiments, the topical analgesic spray composition comprises 0.22 wt. % of one or more essential oils.

In some embodiments wherein the topical analgesic spray composition comprises the organoleptic composition provided herein, the topical analgesic spray concentrate and topical analgesic spray composition comprise linseed oil. In some embodiments, the topical analgesic spray concentrate comprises from 0.02 to 1 wt. %, from 0.02 to 0.1 wt %, or from 0.02 to 0.07 wt. % linseed oil. In certain embodiments, the topical analgesic spray concentrate comprises from 0.02 to 0.07 wt % linseed oil. In still other embodiments, the topical analgesic spray concentrate comprises 0.05 wt. % linseed oil. In some embodiments, the topical analgesic spray composition comprises from 0.005 wt. % to 0.05 wt. %, from 0.004 to 0.2 wt. %, from 0.004 to 0.02 wt %, or from 0.004 to 0.014 wt. % linseed oil. In certain embodiments, the topical analgesic spray composition comprises from 0.004 to 0.014 wt. % linseed oil. In still other embodiments, the topical analgesic spray composition comprises 0.01 wt. % linseed oil.

### Aerosol Spray Dispenser and Methods of Preparing and Using Analgesic Spray Compositions

Provided below is a discussion of aerosol spray dispensers for the topical analgesic spray compositions and methods of preparing and using the topical analgesic spray compositions according to the embodiments provided herein.

### Methods of Preparing Topical Analgesic Spray Concentrates Compositions

Provided herein are methods of preparing topical analgesic spray concentrates and/or topical analgesic spray compositions according to embodiments provided herein.

In some embodiments, provided herein is a method of preparing a topical analgesic spray composition, comprising preparing a mixture comprising a solvent and film-forming agent; adding menthol and camphor to the mixture to form a topical analgesic spray concentrate; and combining the topical analgesic spray concentrate with one or more propellants to provide the topical analgesic spray composition.

In some embodiments, the menthol and camphor may be added individually or in combination. In some embodiments wherein the menthol and camphor are added individually, the menthol may be added first. In other embodiments, the camphor may be added first. In other embodiments wherein the menthol and camphor are added in combination, a menthol/camphor melt can be prepared. In particular, the menthol and/or camphor can be heated in a water bath at a temperature from 30 to 50° C. or from 35 to 45° C. In some embodiments, the temperature may be more than 30° C., more than 35° C., or more than 40° C. In some embodiments, the temperature may be less than 50° C., less than 45° C., or less than 40° C. The menthol and/or camphor may be heated until melted into a colorless liquid, at which time the melt may be removed from the heat.

Additional active ingredients may be added to the topical analgesic spray concentrates and topical analgesic spray compositions described herein. In some embodiments, histamine dihydrochloride, methyl salicylate, methyl nicotinate, and/or capsaicin (if used) may be mixed into the solvent. The histamine dihydrochloride and solvent may be mixed using a suitable mixer or agitator.

In some embodiments, the excipients (including, for example, any sensates, essential oils or mixtures thereof, film-forming agent(s), emollients (e.g., linseed oil, dimethylisosorbide, propanediol), and fragrance) may be added. In certain embodiments, the excipients may be added before or after the addition of camphor and menthol. In other embodiments, the excipients may be added individually or in combination. In certain embodiments, a selection of the excipients may be pre-mixed prior to addition. The excipients may be mixed into the solvent until the solution is visibly clear and no particulate matter remains.

In some embodiments, the prepared menthol/camphor melt may be added to the mixer. If the menthol/camphor melt has begun recrystallizing, the melt may be reheated prior to adding to the mixer. The menthol/camphor melt may be added and mixed with the components of steps two and three until a visibly clear solution is achieved.

Any remaining ingredients of the topical analgesic may be added to the mixer and mixed until uniform. Once uniform, solvent may be used to QS the mixture if necessary.

Process parameters that may be optimized during the various mixing steps can include but are not limited to temperature at which the mixtures are maintained during mixing, temperatures of the ingredients being added to the mixture, duration of mixing, time between addition of ingredients and/or order of mixing/adding ingredients. For example, in some embodiments, the individual mixing steps may be independently carried out at a temperature between 20° C. and 60° C. In other embodiments, the individual mixing steps may be independently carried out for at least 5 minutes, at least 10 minutes, at least 15 minutes, or at least 20 minutes.

In some embodiments, the topical analgesic spray concentrate may be combined with one or more propellants (e.g., isopentane, isobutane, and propane) to provide a topical analgesic spray composition. In certain embodiments wherein the topical analgesic spray concentrate is combined with one or more propellants to provide a topical analgesic spray composition, the topical spray composition comprises 75 wt. % or more of one or more propellants. In some embodiments, the topical analgesic spray composition is packaged in an aerosol spray dispenser.

In some embodiments, the step of combining the topical analgesic spray concentrate with the one or more propellants may be performed at the same time as the packaging step. For example, in some embodiments, the topical analgesic spray concentrate is placed inside the aerosol spray dispenser and the one or more propellants injected into the aerosol spray dispenser containing the topical analgesic spray concentrate. In certain embodiments, the dispenser (valve) is sealed after the topical analgesic spray concentrate is placed inside the dispenser and before the one or more propellants are injected. In some embodiments, the one or more propellants are injected into the dispenser individually. In other embodiments, the one or more propellants are combined and then injected into the dispenser.

### Aerosol Spray Dispenser and Methods of Using Topical Analgesic Spray Composition

In another aspect, the present disclosure provides a pressurized aerosol spray dispenser, containing a topical analgesic spray composition as described herein. In some embodiments, the topical analgesic spray compositions of the present disclosure may be dispensed from pressurized aerosol spray dispensers (or containers) such as cans (canisters) and bottles.

The pressurized aerosol spray dispenser may be manufactured from particular materials or prepared to particular specifications to achieve certain characteristics for storage and dispensing. The pressurized aerosol dispenser may be constructed out of material suitable to withstand particular pressure ranges or temperature ranges that may be observed under various storage conditions. For example, in some embodiments, the pressurized aerosol spray dispenser is selected to withstand an internal pressure of up to 75 psig or exposure to temperatures between 0° F. and 130° F. The ability of a dispenser or container to withstand certain pressures or exposure to particular temperatures may be characterized by observations of any rupturing or deformation of the container.

In some embodiments, the aerosol spray dispenser is constructed out of a metal or metal alloy. In certain embodiments, the aerosol spray dispenser is constructed out of steel or aluminum. In some embodiments wherein the aerosol spray dispenser is constructed out of a metal or metal alloy, the dispenser has an inert interior coating to protect the base metal from corrosion when in contact with the spray composition (concentrate and propellant).

Other considerations for a suitable aerosol spray dispenser may include the combination of the body of the dispenser, the valve and actuator to be used. For example, the individual components of the aerosol spray dispenser may be selected such that when combined with the topical analgesic spray composition (concentrate and propellant) that a desirable spray pattern is delivered to the consumer's body part. For example, in some embodiments, a suitable actuator may include an actuator that provides a particular spray pattern to provide maximum concentration of spray on the consumer's body and to minimize excessive fly-away.

In some embodiments, the aerosol spray dispenser has a delivery spray rate. In certain embodiments, the delivery spray rate is between 0.50 g/sec and 1.00 g/sec. In other embodiments, the delivery spray rate is between 0.50 g/sec and 0.75 g/sec.

In yet another aspect, the present disclosure provides a method for treating muscle and joint ache or pain, comprising administering to a patient in need thereof a topical analgesic spray composition as described herein.

In some embodiments, the method comprises administering the topical analgesic spray composition to the patient's skin at the site of the muscle and joint ache or pain. In other embodiments, the method comprises applying the topical analgesic spray concentrate to the patient's skin at the site of the muscle and joint ache or pain. In certain embodiments, the muscle and joint ache or pain are associated with arthritis, backache, muscle strains, sprains, bruises or cramps.

As described herein, it should be recognized that the topical analgesic spray composition as dispensed from an aerosol container arrives at the site of application on the patient's skin after flash evaporation of the propellants. Thus, it should be acknowledged that the administration of the topical analgesic spray composition may also be considered as applying the topical analgesic spray concentrate to the patient's skin. Accordingly, in one aspect, provided herein is a method for treating muscle and joint ache or pain, comprising applying a topical analgesic spray concentrate as described herein.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “X” includes description of “X”.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EXAMPLES

The presently disclosed subject matter will be better understood by reference to the following Examples, which are provided as exemplary of the invention, and not by way of limitation.

### Example 1: Preparation of a Topical Analgesic Spray Composition

Table 1 shows the individual components in an exemplary formulation of the topical analgesic spray concentrate (without propellant) and topical analgesic spray composition (concentrate with propellant) by percentage of the total net weight. In the following Example, the topical analgesic spray concentrate and topical analgesic spray composition were prepared according to the weight percentages in Table 1.

As described above, following release of the topical analgesic composition from an aerosol spray dispenser, the propellants included in the composition (isopentane, propane, isobutane) flash evaporate, thereby delivering the menthol and camphor to the skin at the concentrations as provided in the concentrate, e.g., 16.0 wt. % menthol and 5.50 wt. % camphor below.

TABLE 1

| | International Nomenclature | Concentrate | Composition |
|---|---|---|---|
| | of Cosmetic Ingredient | (without | (with |
| Ingredient | (INCI) Name | propellant) | propellant) |
| Menthol | Menthol | 16.0% | 3.20% |
| Camphor | Camphor | 5.50% | 1.10% |
| Isopentane | Isopentane | 0.00% | 55.0% |
| A-70 | Propane, Isobutane | 0.00% | 25.0% |
| SDA 40 B | Ethanol, denatured | 64.7% | 12.94% |
| Coolact ® 10 | Menthoxypropanediol | 5.00% | 1.00% |
| Coolact ® P | Isopulegol | 5.00% | 1.00% |
| Essential Oil | Peppermint Oil, Eucalyptus | 1.10% | 0.22% |
| blend | Oil, Rosemary Oil, Spanish | | |
| | Marjoram Oil, Clove Oil, | | |
| | Frankincense, Vitamin E | | |
| Zemea Propane | Propanediol | 1.00% | 0.20% |
| Diol | | | |
| Fragrance | Mystic Sage and Minerals | 1.00% | 0.20% |
| DMI | Dimethyl Isosorbide | 0.50% | 0.10% |
| Advantage ™ | Vinyl caprolacta/VP/ | 0.10% | 0.020% |
| LC-A | dimethylamino ethyl | | |
| | methacrylate copolymer | | |
| Hotact ® VBE | Vanillyl butyl ether | 0.050% | 0.010% |
| Linseed Oil | Linseed oil | 0.050% | 0.010% |

Topical Analgesic Spray Concentrate Preparation.

Denatured ethanol was agitated at 25° C. in a propeller agitator. To the ethanol, Advantage™ LC-A was added slowly and mixed until all solids were dissolved and the mixture appeared uniform (≥5 minutes).

While the ethanol mixture was continuously mixed and maintained at the same temperature, camphor was added and mixed until all particles were dissolved (≥15 minutes). After camphor was added and fully dissolved, menthol was added to the mixture and mixed until all solids were dissolved (≥15 minutes).

In a separate vessel, Coolact® P, Coolact® 10, Hotact® VBE, dimethyl isosorbide, and propanediol were combined and mixed until a uniform mixture was obtained. This mixture was then added to the ethanol mixture containing camphor, menthol and Advantage™ LC-A. The ethanol mixture was mixed until homogenous (≥5 minutes). While mixing at a moderate speed, the essential oil blend, fragrance and linseed oil were added sequentially to the ethanol mixture until a final homogenous concentrate was obtained.

Topical Analgesic Spray Composition Preparation.

After mixing as described previously, the concentrate was introduced into the desired package in the desired ratio. The valve was then permanently sealed onto the container by first pulling a vacuum on the package and then immediately crimping (sealing) the valve to the container using the predetermined crimp depth and crimp diameter values to insure a leak-free seal.

The desired amount of isopentane was added directly by forcing the liquid under pressure through and around the stem of the valve. In the same manner, the desired amount of the propane/isopentane blend was added to the package. Alternatively, a blend of the isopentane, isobutane, and propane may be premixed and added in a single step.

The filled unit was passed through a heating cycle (water bath) where the internal temperature was raised to 130° F. in order to comply with DOT regulations for shipping

### Example 2: Evaluation of Drying Time for a Topical Analgesic Spray Composition

This example describes efforts to evaluate the drying time of the topical analgesic spray composition of Example 1, as compared to other commercially available mentholated spray medications, IcyHot® Dry Spray and BioFreeze™ Spray—Fast Acting Menthol Pain Relief”. Mass loss of a fixed quantity of each of the spray products over time was observed and evaluated as a proxy for volatility (i.e., quick drying time).

The spray composition described in Table 1 of Example 1 was evaluated in this study. The ingredient listing for the two comparison spray medications are as follows: for BioFreeze Spray: Menthol (10.5%), alcohol denatured, Arnica Montana Flower Extract, Calendula Officinalis Flower Extract, Camellia Sinensis Leaf Extract, Chamomilla Recutita (Matricaria) Flower Extract, Dimethyl Sulfone (MSM), Echinacea Angustifolia Extract, Ilex Paraguariensis Leaf Extract, Isopropyl Myristate, Juniperus Communis Fruit Extract, Water; and for IcyHot® Dry Spray: Menthol (16%), alcohol denatured (55%), glycerin, isobutene, propylene glycol, water.

The products to be evaluated were each sprayed in a plastic weigh boat placed on an analytical balance (PD561, Mettler Toledo) and the apparent mass loss observed over five minutes at constant room temperature (75° F.) and relative humidity (30%). It was expected that the product having the greatest volatility would lose the most mass in a fixed amount of time of five minutes. This method eliminated dose variation of products upon actuation (i.e., more products will take more time to dry) to skin in a typical clinical study and ensured consistency in measuring drying time based upon rate of evaporation by recording a loss in weight in a fixed time of 5 minutes.

In order to observe mass loss, each product was individually evaluated over three separate trials. The product spray for each trial was sprayed into plastic weigh boat placed on an analytical balance, as described above, at a perpendicular distance of 6 inches between the nozzle of the sprayer and the weigh boat (spray area 4×4 square inches), to provide mass exceeding one gram (>1 g). The product spray was allowed to dry until a mass of 1 g was reached, at which point the five-minute observation period began (i.e., the initial mass of the spray was fixed at 1 g and recorded at t=0 minutes). One gram was selected as the initial mass based upon typical amount sprayed in actual application by consumers. After 5 minutes, the mass left behind in the plastic weight boat was recorded at t=5 minutes. The difference in weight gave the mass of spray that had volatilized over the five-minute evaluation period. The three final masses at t=5 minutes were averaged to provide an average final mass, from which an average mass loss and a drying rate per minute could be calculated.

TABLE 2
Drying rate of products tested

| | | Initial | | | | |
|---|---|---|---|---|---|---|
| Item | | weight at | Final weight at t = 5 | | Loss of mass | Drying |
| # | Product | t = 0 min | mins | | in 5 mins | rate/min |
| 1 | Icy Hot ® | 1.000 g | 0.860 g | Average: | Loss in mass | 0.128 g/5 mins = |
| | Spray | 1.000 g | 0.881 g | 0.872 g | (1 − 0.872) g = | 0.026 g/min |
| | | 1.000 g | 0.875 g | | 0.128 g | |
| 2 | BioFreeze ™ | 1.000 g | 0.869 g | Average: | Loss in mass | 0.128 g/5 mins = |
| | Spray | 1.000 g | 0.871 g | 0.872 g | (1 − 0.872) g = | 0.026 g/min |
| | | 1.000 g | 0.875 g | | 0.128 g | |
| 3 | Example 1 | 1.000 g | 0.432 g | Average: | Loss in mass | 0.562 g/5 mins = |
| | Dry Spray | 1.000 g | 0.409 g | 0.438 g | (1 − 0.438) g = | 0.112 g/min |
| | Formulation | 1.000 g | 0.475 g | | 0.562 g | |

The dry spray of Example 1 demonstrated the fastest drying rate of 0.112 g/min, exceeding the observed drying rate of the competitor sprays by an approximate factor of 4 (0.112/0.026). In addition, Icy Hot® and BioFreeze™ exhibited a dripping effect as liquid ran out from the area after spraying. The dry spray of Example 1 did not leave any liquid droplets as residue and dried almost instantaneously.

What is claimed is:

1. A topical analgesic spray composition, comprising:
2 to 4 wt. % menthol;
1 to 3 wt. % camphor,
10 wt. % or more ethanol; and
75 wt. % or more of one or more propellants.

2. The topical analgesic spray composition of claim 1, further comprising 0.005 to 0.050 wt. % histamine dihydrochloride.

3. The topical analgesic spray composition of claim 1, wherein the 2 to 4 wt. % menthol and the 1 to 3 wt. % camphor form a eutectic mixture.

4. The topical analgesic spray composition of claim 1, wherein the composition has a combined concentration of menthol and camphor of 4 wt. % or more.

5. The topical analgesic spray composition of claim 1, wherein the one or more propellants comprises isopentane, propane, or isobutane, or any combinations thereof.

6. The topical analgesic spray composition of claim 5, wherein topical analgesic spray composition comprises 50 to 70 wt. % isopentane.

7. The topical analgesic spray composition of claim 5, wherein the topical analgesic spray composition comprises 10 to 30 wt. % of a mixture of propane and isobutane.

8. The topical analgesic spray composition of claim 1, wherein the analgesic spray composition comprises between 10 and 15 wt. % ethanol.

9. The topical analgesic spray composition of claim 1, further comprising 0.01 to 0.2 wt. % film-forming agent.

10. The topical analgesic spray composition of claim 9, wherein the film-forming agent comprises a terpolymer of vinylpyrrolidone, vinyl caprolactum and dimjethylaminoethyl methacrylate.

11. The topical analgesic spray composition of claim 1, further comprising 1 to 3 wt. % of one or more sensates.

12. The topical analgesic spray composition of claim 11, wherein the one or more sensates includes one or more of menthoxypropanediol, isopulegol, and vanillyl butyl ether.

13. The topical analgesic spray composition of claim 1, further comprising 0.1 to 0.6 wt. % of one or more essential oils.

14. The topical analgesic spray composition of claim 13, wherein the one or more essential oils comprises one or more of peppermint (Mentha piperita) oil, eucalyptus (Eucalyptus globulus) oil, rosemary (Rosmarinus officinalis) oil, clove (Eugenia caryophyllata) oil, Spanish marjoram (Thymus mastichina) oil and frankincense (Olibanum or Boswellia carterii) oil.

15. The topical analgesic spray composition of claim 1, further comprising 0.005 to 0.05 wt. % linseed oil.

16. The topical analgesic spray composition of claim 1, comprising 0.1 to 0.4 wt. % fragrance.

17. The topical analgesic spray composition of claim 1, wherein the spray composition has a drying rate of at least 0.05 g/min as determined by gravimetric evaluation at a temperature of 75° F.±10° F. and relative humidity of 30%±10%.

18. An aerosol spray dispenser, comprising a topical analgesic spray composition, wherein the topical analgesic spray composition comprises:
2 to 4 wt. % menthol;
1 to 3 wt. % camphor,
10 wt. % or more ethanol; and
75 wt. % or more of one or more propellants.

19. The aerosol spray dispenser of claim 18, wherein the dispenser is a pressurized bottle or can.

20. A topical analgesic spray concentrate comprising:
10 to 16 wt. % menthol;
5 to 11 wt. % camphor;
0.05 to 1 wt. % film-forming agent; and
50 to 70 wt. % ethanol.

21. The topical analgesic spray concentrate of claim 20, further comprising 0.025 to 0.250 wt. % histamine dihydrochloride.

22. The topical analgesic spray concentrate of claim 20, wherein the 10 to 16 wt. % menthol and the 5 to 11 wt. % camphor form a eutectic mixture.

23. The topical analgesic spray concentrate of claim 20, wherein the topical analgesic spray concentrate has a combined concentration of menthol and camphor of 20 wt. % or more.

24. The topical analgesic spray concentrate of claim 20, wherein the film-forming agent comprises a terpolymer of vinylpyrrolidone, vinyl caprolactum and dimethylaminoethyl methacrylate.

25. The topical analgesic spray concentrate of claim 20, further comprising 5 to 15 wt. % of one or more sensates.

26. The topical analgesic spray concentrate of claim 25, wherein the one or more sensates includes one or more of menthoxypropanediol, isopulegol, and vanillyl butyl ether.

27. The topical analgesic spray concentrate of claim 20, further comprising 0.5 to 3 wt. % of one or more essential oils.

28. The topical analgesic spray concentrate of claim 27, wherein the one or more essential oils comprises one or more of peppermint (Mentha piperita) oil, eucalyptus (Eucalyptus globulus) oil, rosemary (Rosmarinus officinalis) oil, clove (Eugenia caryophyllata) oil, Spanish marjoram (Thymus mastichina) oil, and frankincense (Olibanum or Boswellia carterii) oil.

29. The topical analgesic spray concentrate of claim 20, further comprising 0.02 to 1 wt. % linseed oil.

30. The topical analgesic spray concentrate of claim 20, comprising 0.5 to 2.0 wt. % fragrance.

31. A method of preparing a topical analgesic spray composition, the method comprising:
preparing a mixture comprising a solvent and a film-forming agent;
adding menthol and camphor to the mixture comprising the solvent and the film-forming agent to form a topical analgesic spray concentrate; and
combining the topical analgesic spray concentrate with one or more propellants to provide the topical analgesic spray composition,
wherein the topical analgesic spray composition comprises 75 wt. % or more of one or more propellants.

32. The method of claim 31, wherein preparing a mixture comprising menthol and camphor comprises preparing a eutectic mixture comprising menthol and camphor.

33. The method of claim 31, comprising adding one or more of histamine dihydrochloride, sensates, and essential oils to the mixture comprising menthol and camphor.

34. The method of claim 31, wherein the step of combining the topical analgesic spray concentrate and the one or more propellants to provide the topical analgesic spray composition comprises placing topical analgesic spray concentrate inside an aerosol spray dispenser,
and injecting one or more propellants into the aerosol spray dispenser containing the topical analgesic spray concentrate.

35. The method of claim 34, wherein the aerosol spray dispenser is sealed after the topical analgesic spray concentrate is placed inside the dispenser and before the one or more propellants are injected.
